(19) United States
(12) Patent Application Publication
(10) Pub. No.: US 2017/0014030 A1
(43) Pub. Date: Jan. 19, 2017

(54) Endoscope Featuring Depth Ascertainment

(71) Applicant: Siemens Aktiengesellschaft, Muenchen (DE)

(72) Inventors: Peter Rentschler, Neuhengstett (DE); Anton Schick, Velden (DE)

(73) Assignee: Siemens Aktiengesellschaft, Muenchen (DE)

(21) Appl. No.: 15/124,136

(22) PCT Filed: Feb. 26, 2015
(86) PCT No.: PCT/EP2015/054036

§ 371 Date: Sep. 7, 2016

(30) Foreign Application Priority Data
Mar. 7, 2014 (DE) 10 2014 204 244.5

(51) Int. Cl. A61B 5/00; A61B 1/00; A61B 1/018; A61B 1/06; A61B 1/313; A61B 1/07; A61B 1/05; A61B 5/107

(52) U.S. Cl. CPC A61B 5/0084; A61B 1/051; A61B 1/00096; A61B 5/1076; A61B 5/1079; A61B 1/0638; A61B 1/3132; A61B 1/063; A61B 1/0684; A61B 1/07; A61B 1/018

(57) ABSTRACT

An endoscope for determining the depth of a subregion of a cavity may include at least one imaging channel having a first optical axis, with at least one first optical deflection device arranged in the at least one imaging channel. The optical deflection device may be designed to transversely offset the first optical axis parallel to the first optical axis.

### CROSS-REFERENCE TO RELATED APPLICATIONS

This application is a U.S. National Stage Application of International Application No. PCT/EP2015/054036 filed Feb. 26, 2015, which designates the United States of America, and claims priority to DE Application No. 10 2014 204 244.5 filed Mar. 7, 2014, the contents of which are hereby incorporated by reference in their entirety.

### TECHNICAL FIELD

The invention relates to an endoscope for determining the depth of a partial region of a cavity.

### BACKGROUND

In recent years, the number of minimally invasive surgeries has increased steadily. In minimally invasive surgery, endoscopes (3D endoscopes) are used which enable depth determination of a cavity to be examined in a patient and simultaneously enable an imaging method. According to the prior art, a plurality of ports are placed for determining the depth of the cavity, for example an abdominal cavity of the patient.

The ports leading to the cavity (observation space) which are configured as access channels are typically very narrow. This is the case because the goal of minimally invasive surgery is to operate on the patient in as gentle a fashion as possible. Due to the narrow channels, the design of 3D endoscopes is very restricted. 3D endoscopes that are known from the prior art are here configured as a cylindrical, longitudinal tube.

For determining the depth of the cavity by means of a 3D endoscope, further optical components that enable active or passive triangulation of the cavity and thus permit depth determination are provided according to the prior art. Critical for the resolution of the depth determination in the active or passive triangulation is the size of a triangulation base. The triangulation base here designates the distance between a projector and an optical imaging system in the endoscope. The larger the triangulation base, the better the resolution of the depth determination.

In order to achieve imaging performance that suffices for minimally invasive surgery, optical imaging systems which have a relatively large cross section are typically used according to the prior art. Since a sufficiently large imaging performance is indispensable in minimally invasive surgery, the triangulation base must therefore be correspondingly reduced, which thus reduces the resolution of the depth determination.

### SUMMARY

One embodiment provides an endoscope for determining the depth of a partial region of a cavity, which comprises at least a first imaging channel having a first optical axis, wherein arranged within the first imaging channel is at least a first optical deflection apparatus which is configured for causing a shift of the first optical axis in a manner that is transverse parallel with respect to the first optical axis.

In one embodiment, the first optical deflection apparatus is arranged at a distal end of the endoscope.

In one embodiment, the first imaging channel has an objective lens, wherein the objective lens comprises the first optical deflection apparatus.

In one embodiment, the first imaging channel comprises at least one lens.

In one embodiment, the endoscope includes at least one relay lens.

In one embodiment, the first optical deflection apparatus is configured as a parallelepiped.

In one embodiment, the first optical deflection apparatus has at least two mirrored internal surfaces.

In one embodiment, the endoscope includes a projection channel, wherein the projection channel comprises a projection apparatus which is configured for projecting a pattern onto a surface of the partial region of the cavity.

In one embodiment, the projection apparatus comprises a diffractive optical element for producing the pattern.

In one embodiment, the pattern is a color-coded color pattern.

In one embodiment, the projection channel is optically coupled to a light source.

In one embodiment, the endoscope includes an instrumentation channel.

In one embodiment, the include a second imaging channel having a second optical axis, wherein arranged within the second imaging channel is a second optical deflection apparatus which is configured for causing a shift of the second optical axis in a manner that is transverse parallel with respect to the second optical axis.

In one embodiment, the direction of the transverse parallel shift of the second optical axis is the opposite of the direction of the transverse parallel shift of the first optical axis.

In one embodiment, the second imaging channel is configured as a projection channel.

In one embodiment, the endoscope has an observation angle of 30°.

### BRIEF DESCRIPTION OF THE DRAWINGS

Example aspects and embodiments of the invention are described below with reference to the figures, in which:

FIG. 1 shows a schematic sectional view of a first imaging channel comprising a first optical deflection apparatus;

FIG. 2 shows a further schematic sectional view of a first imaging channel comprising relay lenses;

FIG. 3 shows a schematic sectional view of an endoscope having a first and a second imaging channel;

FIG. 4 shows an enlarged diagram of the endoscope shown in FIG. 3; and

FIG. 5 shows a schematic sectional view of an endoscope comprising a first imaging channel and a projection channel.

### DETAILED DESCRIPTION

Embodiments of the present invention provide an improved optical depth determination of an endoscope.

Some embodiments provide an endoscope for determining the depth of a partial region of a cavity, which endoscope comprises at least a first imaging channel having a first optical axis, wherein arranged within the first imaging channel is at least a first optical deflection apparatus which is configured for causing a shift of the first optical axis in a manner that is transverse parallel with respect to the first optical axis.

The endoscope may comprise a first optical deflection apparatus which shifts a first optical axis of the first imaging channel in a transverse parallel manner.

The first optical axis of the imaging channel can be an axis of symmetry of a reflective or refractive optical element. If the imaging channel comprises a lens system and/or imaging system, the first optical axis is that optical axis which is formed by the optical axis of the individual optical elements.

Advantageously, the transverse parallel shift of the first optical axis according to the invention by means of the first optical deflection apparatus permits enlargement of a triangulation base of the endoscope according to the invention. The transverse parallel shift of the first optical axis should here expediently be such that it results in an enlargement of the triangulation base. By enlarging the triangulation base of the endoscope, the resolution of the depth determination (depth resolving power) is advantageously improved. In particular, even though the triangulation base is enlarged, the endoscope and/or the first imaging channel do not have to be reduced in size with respect to endoscopes known from the prior art in order to improve the depth resolution. As a result, the imaging performance of known endoscopes is advantageously not impaired.

The transverse parallel nature of the shift of the first optical axis is to be understood to be approximate. Critical is that the shift which is effected by means of the first optical deflection apparatus is used to enlarge the triangulation base. In other words, the first optical deflection apparatus is configured for enlarging the triangulation base.

The first optical deflection apparatus is preferably arranged at a distal end of the endoscope.

A beam diameter of a beam entering the imaging channel at the distal end of the endoscope is typically small, because strong focusing of light rays that make up the beam takes place upon entry into the first imaging channel of the endoscope. As a result, the required space for the first optical deflection apparatus is advantageously reduced.

According to one embodiment, the first imaging channel has an objective lens, wherein the objective lens comprises the first optical deflection apparatus.

The first optical deflection apparatus, based on a ray that is incident in the first imaging channel, may be arranged downstream of a first lens of the objective lens. An objective lens may be configured as a wide-angle objective lens. Here, a beam entering the first imaging channel is focused in a pupil. Advantageously, the first optical deflection apparatus is arranged in a region of the pupil such that as a result, the first deflection apparatus can be configured to be small with respect to its geometric extensions, since the pupil of the beam that is incident in the first imaging channel is likewise small.

According to one embodiment, the first imaging channel comprises further lenses.

The first imaging channel may comprise a lens system which comprises a plurality of lenses. By arranging at least one lens in the imaging channel, an optical imaging system is arranged in the first imaging channel. The imaging of the partial region of the cavity by the first imaging channel is here effected by means of the lens or by means of the optical imaging system. By way of example, the lens can be configured as a collimator, dispersing lens or a focusing lens. Further optical components provided can be for example mirrors, glasses, crystals, beam splitters, Faraday isolators and/or prisms.

By way of example, an additional angular change of the first optical axis can be provided by means of said optical components. The angular change preferably ranges from 1° to 5°, with an angular change of less than or equal to 3° being particularly preferred.

The first imaging channel of the endoscope preferably comprises a further lens which is configured as a relay lens.

Relay lenses are typically used to transmit the image from the distal end of the endoscope to a further end of the endoscope that is situated opposite the distal end.

According to one embodiment, the first optical deflection apparatus is configured as a parallelepiped.

The parallelepiped or the first optical deflection apparatus is arranged here such that the transverse parallel shift of the beam is the result of reflections within the parallelepiped of a beam that is incident in the parallelepiped. In other words, the first optical deflection apparatus is configured as a type of prism block, wherein the beam that is incident in the first imaging channel at the distal end of the endoscope is shifted in a transverse parallel manner by way of two reflections at internal surfaces of the first optical deflection apparatus, in particular by two total internal reflections of the incident beam. Owing to the transverse parallel shift of the light beam, the triangulation base of the endoscope is advantageously increased in size, as a result of which the resolution of the depth determination is improved. The transverse parallel shift of the light beam here corresponds to the transverse parallel shift of the first optical axis.

Some embodiments include a first optical deflection apparatus, e.g., a parallelepiped, which has at least two mirrored internal surfaces.

By means of the mirrored internal surfaces of the first optical deflection apparatus, the light rays entering the first imaging channel are reflected at least twice within the first optical deflection apparatus, in particular by way of total internal reflection. As a result, the transverse parallel shift of the first optical axis is made possible by means of the first optical deflection apparatus. Provision is made here for further optical components, for example lenses and/or objective lenses, to be arranged upstream of the first optical deflection element with respect to the light rays entering the first imaging channel. In a particularly efficient embodiment, the first optical deflection apparatus comprises only two individual mirrors which form two sides of an imaginary parallelepiped.

According to one embodiment, the endoscope comprises a projection channel, wherein the projection channel comprises a projection apparatus which is configured to project a pattern onto a surface of the partial region of the cavity.

Active triangulation of the partial region of the cavity is advantageously made possible by the arrangement of at least one projection channel in the endoscope. Structured light, in other words a pattern, is projected here onto a surface of the partial region of the cavity by means of the projection apparatus which is arranged in the projection channel. A correspondence problem in the active triangulation is advantageously mitigated or even entirely resolved by means of the projected pattern, in particular by means of a coded pattern.

A projection apparatus which comprises a diffractive optical element for producing the pattern is particularly preferred.

A DOE projector is advantageously implemented by a projection apparatus comprising the diffractive optical element. A DOE projector is here considered to be a projection apparatus which comprises a diffractive optical element (DOE in short). Since DOE projectors require less space than projectors which typically have a slide for producing the pattern, the projection channel can be configured with a comparatively small diameter or with a comparatively small cross-sectional area. The cross-sectional area of the projection apparatus or of the projection channel is in particular less than or equal to 2 mm². Space-saving active triangulation of the partial region of the cavity is made possible overall by the projection channel, the first imaging channel and the projection apparatus which is arranged in the projection channel and comprises a diffractive element.

Some embodiments provide active triangulation effected by a color-coded pattern.

In other words, the endoscope permits active color-coded triangulation of the partial region of the cavity.

In one embodiment, the projection channel is optically coupled to a light source.

In some embodiments, light source may be a laser or a light-emitting diode (LED). A single-mode fiber can here be provided for optically coupling the projection channel to the light source, in particular to the laser. Exactly one light mode, the base mode, is guided in the single-mode fiber advantageously such that interference between a plurality of light modes that could result in disturbance of the projected pattern is avoided.

The light from the light source, in particular the laser, is thus introduced in the projection channel by means of the single-mode fiber. The wavelength of the laser for optimum dot contrast generation, for example in the blue spectral range, can here be adapted to the use in minimally invasive surgery. What is particularly preferred is that a disturbing influence of the daylight and/or artificial light due to the use of a laser as the light source is reduced for example using an interference filter.

According to one embodiment, the endoscope comprises an instrumentation channel.

Surgical tools required for minimally invasive surgery can be advantageously introduced into the cavity by means of the instrumentation channel. Installation space is saved due to the arrangement of a diffractive optical element in the projection channel and can in turn be used for the instrumentation channel. In particular, a plurality of instrumentation channels can be provided.

In one embodiment, the endoscope comprises a second imaging channel which extends parallel to the first imaging channel and has a second optical axis, wherein arranged within the second imaging channel is a second optical deflection apparatus which is configured for causing a shift of the second optical axis in a manner that is transverse parallel with respect to the second optical axis.

Advantageously, stereoscopy of the partial region of the cavity is made possible by the second imaging channel which has a second optical deflection apparatus. What is particularly advantageous is that, due to the first and the second optical deflection apparatus, the triangulation base is enlarged as compared to a prior art endoscope for stereoscopy. As a result, the resolution of the depth determination of the partial region of the cavity is advantageously improved by way of the endoscope that is proposed here. Here, a second imaging channel which is configured corresponding to the first imaging channel is provided.

Some embodiments include a second imaging channel whose second optical deflection apparatus has a direction of the transverse parallel shift that is the opposite of the direction of the transverse parallel shift of the first optical axis.

As a result, the triangulation base is advantageously further enlarged, with the result that the resolution of the depth determination is further improved.

In one embodiment, the second imaging channel is configured in the form of a projection channel.

In general, each imaging channel can be used as a projection channel. Active triangulation of the partial region of the cavity is advantageously made possible by the projection channel. If the endoscope has two imaging channels and one projection channel, active stereoscopy of the partial region of the cavity can be effected with the endoscope.

According to one embodiment, the endoscope has an observation angle of 30°.

Here, provision is made for an arrangement of the first optical deflection apparatus within the endoscope which has an observation angle of 30° (30° endoscope).

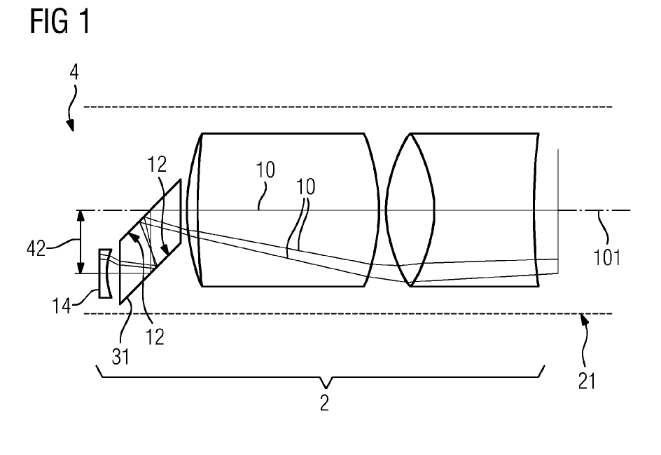

FIG. 1 schematically illustrates a first imaging channel 21 of an endoscope 1 (not depicted). An objective lens 2 is arranged here in the first imaging channel 21, which objective lens 2 has a first optical axis 101. A first optical deflection apparatus 31 is provided according to the invention for a transverse parallel shift 42 of the first optical axis 101 of the objective lens 2. The first optical deflection apparatus 31 is arranged here downstream of a first lens 14 of the objective lens 2 with respect to light rays 10 that are incident in the first imaging channel 21. In other words, the first optical deflection apparatus 31 is integrated in the objective lens 2. In the case of integration of the first optical deflection apparatus 31 in the objective lens 2, profiles of incident light rays 10 which have changed because of it must be taken into consideration as well. The objective lens 2 and consequently also the first optical deflection apparatus 31 are arranged at a distal end 4 of the endoscope 1 (not depicted).

The deflection apparatus 31 is used to deflect or shift the light rays 10 (beams) that are incident in the first imaging channel 21 such that a transverse parallel shift 42 of the light rays 10 is preferably produced. In the exemplary embodiment shown in FIG. 1, the first optical deflection apparatus 31 is configured as a parallelepiped and has at least two internal surfaces 12 for deflecting the incident light rays 10. The incident light rays 10 are here reflected at the internal surfaces 12 of the first optical deflection apparatus 31, in particular by way of total internal reflection.

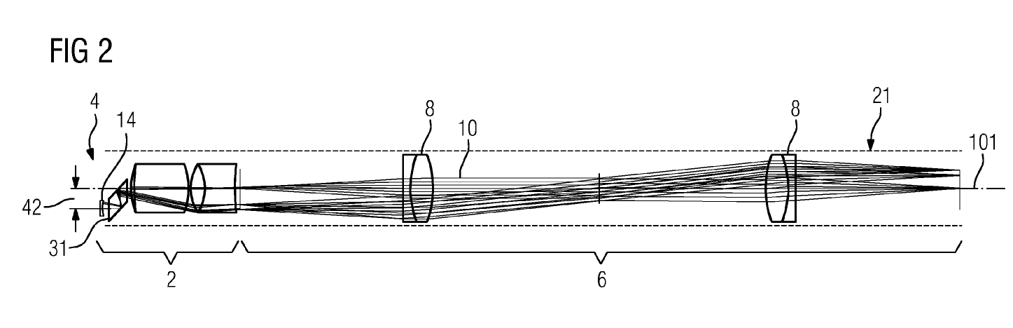

One considerable advantage of the first optical deflection apparatus 31 is that it requires only a small installation space, for example as compared to relay lenses 8 (two shown in FIG. 2). The geometric extensions of the first optical deflection apparatus are in particular smaller than the geometric extensions of typical imaging channels. As a result, the first optical deflection apparatus 31 can be arranged advantageously in existing imaging channels of known endoscopes without unfavorably enlarging the geometric extensions of the imaging channels or of the endoscopes. Conceivable is also an arrangement of the first optical deflection apparatus 31 within an endoscope having an observation angle of 30° (30° endoscopes).

In the exemplary embodiment of the endoscope 1 depicted in FIG. 1, the first optical deflection apparatus 31 is arranged downstream of the first lens 14 of the objective lens 2 with respect to the incident light rays 10. However, it is possible to provide for a first optical deflection apparatus 31 which is not part of the objective lens 2 and is consequently arranged upstream or downstream of the objective lens 2. By way of example, arrangement downstream of the objective lens 2 is advantageous if an exit pupil of the objective lens 2 is located upstream of the objective lens 2 in the distal direction.

Furthermore, a camera, in particular a 3-chip camera, can be provided in the first imaging channel 21. It is possible here to integrate the camera, the objective lens 2 and the first optical deflection apparatus 31 in one chip, with the result that an arrangement is produced that saves the maximum possible installation space.

FIG. 2 shows a further schematic sectional view of a first imaging channel 21 along an axis of symmetry (endoscope axis) of a cylindrical endoscope 1 (not depicted).

A first optical deflection apparatus 31 is arranged within an objective lens 2 at a distal end 4 of the first imaging channel 21 or of the endoscope 1, wherein the first optical deflection apparatus 31 is arranged downstream of a first lens 14 of the objective lens 2 with respect to light rays 10 that enter the first imaging channel 21.

As already depicted in FIG. 1, a transverse parallel shift 42 of an optical axis 101 is made possible by the first optical deflection apparatus 31 which is configured as a parallelepiped. In the exemplary embodiment depicted in FIG. 2, the first optical axis 101 relates to the optical axis of further lenses of the objective lens 2 which are arranged downstream of the first optical deflection apparatus 31 with respect to the entering light rays 10 and/or relates to the optical axis of relay lenses 8 arranged in the imaging channel 21. Here, the relay lenses 8 arranged in the first imaging channel 21 form what is known as a first relay stage 6 of the first imaging channel 21.

The relay lenses 8 and consequently the first relay stage 6 typically have a larger geometric extension than the first optical deflection element 31. In other words, the geometric extension, in particular a diameter of the imaging channel 21, is limited not by the first optical deflection apparatus 31 but by the relay lenses 8 that are arranged in the first imaging channel 21. Consequently, a minimum geometric extension of the first imaging channel 21 is defined by the relay lenses 8 that are arranged in the first imaging channel 21. Advantageously, for arranging the first optical deflection apparatus 31 in the first imaging channel 21, no enlargement of the first imaging channel 21 is therefore necessary.

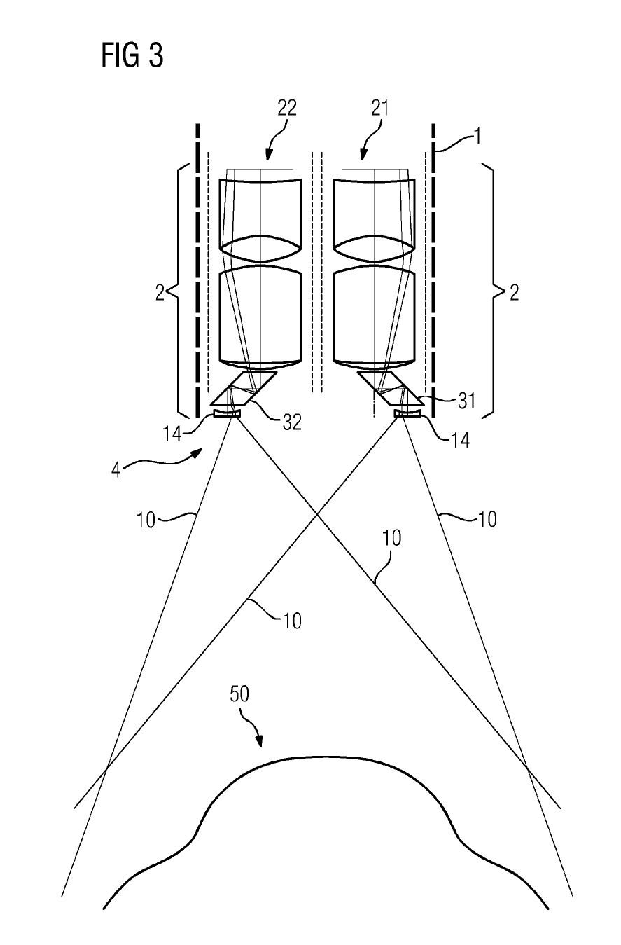

FIG. 3 illustrates a schematic sectional view of an endoscope 1, wherein the endoscope 1 comprises a first imaging channel 21 and a second imaging channel 22.

An objective lens 2 is arranged both in the first and in the second imaging channel 21, 22. Provision may be made here for a camera for recording the images of the first and the second imaging channel 21, 22 to be arranged within said imaging channels 21, 22 and/or to be integrated directly in said objective lenses 2. The first and second imaging channels 21, 22 or the objective lenses furthermore comprise a first and second optical deflection apparatus 31, 32.

A first lens 14 of the respective objective lens 2 is provided in each case upstream of the first and the second optical deflection apparatus 31, 32 with respect to light rays 10 entering the imaging channels 21, 22 at a distal end 4 of the endoscope 1. Here, the first and second imaging channels 21, 22 form an image of a partial region 50 of a cavity from different viewing directions. As a result, stereoscopy of the partial region 50 is advantageously made possible. The first and second deflection apparatuses 31, 32 in the first and second imaging channels 21, 22 are arranged such that in each case a transverse parallel shift of the light rays 10 in the opposite direction results. As a result, a triangulation base (not shown) of the endoscope 1 is advantageously enlarged, as a result of which the resolution of the depth determination of the partial region 50 of the cavity is improved.

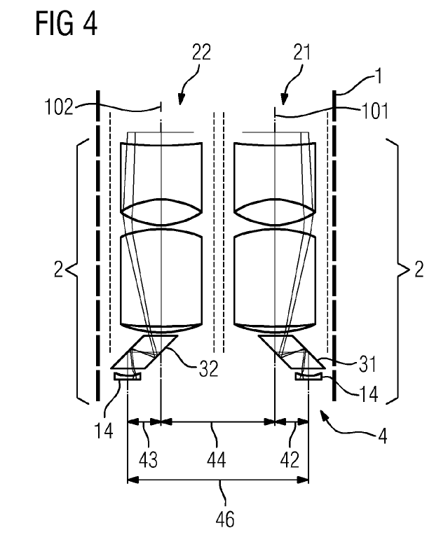

FIG. 4 shows an enlarged illustration of the endoscope 1 depicted in FIG. 3. In each case, an objective lens 2, an optical deflection apparatus 31, 32 and a first lens 14 of the objective lenses 2 are arranged again at a distal end 4 of the endoscope 1 in the first and in the second imaging channel 21, 22. The first imaging channel 21 has a first optical axis 101 and the second imaging channel 22 has a second optical axis 102.

The deflection apparatuses 31, 32 arranged at the distal end 4 permit enlargement of an original triangulation base 44 of known endoscopes, wherein the original triangulation base 44 is defined by the distance between the first and second optical axes 101, 102. The first optical deflection apparatus 31 has a transverse parallel shift 42 of the first optical axis 101 which is in the opposite direction of a transverse parallel shift 43 of the second optical axis 102, wherein the transverse parallel shift 43 of the second optical axis 102 is made possible by means of the second optical deflection apparatus 32. Overall, the result is a triangulation base 46 which is enlarged with respect to the original triangulation base 44. The resolution of the depth determination of the endoscope 1 is thereby advantageously further improved.

If the imaging channels 21, 22 have an additional angular change in their optical axes 101, 102, it is possible by means of the angular change of a beam entering said channels 21, 22 to effect steering of the beam such that chief rays of the beams intersect on an endoscope axis in the center of the objective lenses 2 (pupil). As a result, a shift of the images between the first and the second imaging channels 21, 22 is reduced.

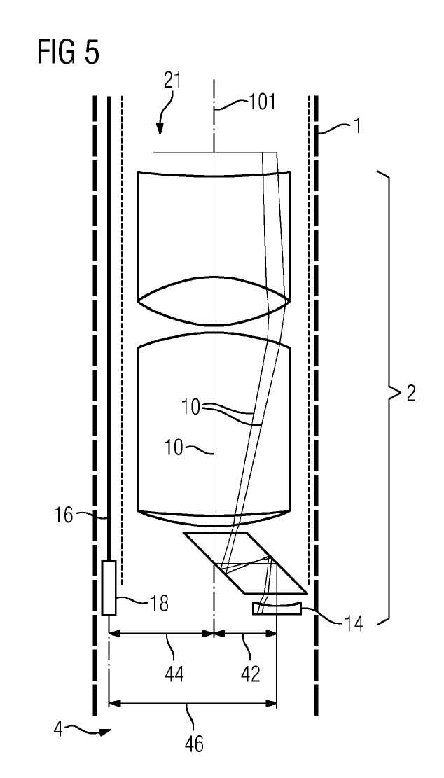

FIG. 5 illustrates a schematic sectional view of an endoscope 1 comprising a first imaging channel 21 and a projection channel 16. Here, a projector 18 is arranged within the projection channel 16, which projector 18 comprises a diffractive optical element (DOE). Such a projector 18 is designated as DOE projector. Active triangulation is made possible thereby by means of a color-coded and/or dot-coded pattern.

Arranged at a distal end 4 of the endoscope 1 is a first deflection apparatus 31 which makes possible an enlarged triangulation base 46. To this end, a first optical axis 101 is shifted 42 transversely. An original triangulation base 44 is thus advantageously enlarged. A first lens 14 of the objective lens 2 is arranged upstream of the first optical deflection apparatus 31 with respect to light rays 10 that enter the first imaging channel 21 at the distal end 4, with the result that the first optical deflection apparatus 31 is arranged between the first lens 14 and further lenses of the objective lens 2. The first optical axis 101 is here defined by said further lenses of the objective lens 2.

The projection channel 16, the first and/or second imaging channels 21, 22 can comprise further optical components, for example lenses, mirrors, gratings, beam splitters and/or prisms and/or entire optical apparatuses, for example further objective lenses. The first and/or second imaging channel 21, 22 can be formed in particular by way of an objective lens. Here, a camera, for example a 3-chip camera, can be arranged at the objective lens 2 and/or integrated in the objective lens 2. The images are here guided preferably via optical fibers, in particular by way of a single-mode fiber.

Even though the invention is illustrated and described in more detail by the preferred exemplary embodiments, the invention is not limited by the disclosed examples, or other variations can be derived therefrom by the person skilled in the art without departing from the scope of protection of the invention.

What is claimed is:

1. An endoscope for determining the depth of a partial region of a cavity, the endoscope comprising:
exactly one imaging channel having a first optical axis,
a first optical deflection apparatus arranged in the first imaging channel, the first optical deflection apparatus configured to cause a shift of the first optical axis in a direction transverse parallel with respect to the first optical axis;
wherein the imaging channel has an objective lens that comprises the first optical deflection apparatus; and
a projection channel having a projection apparatus for active triangulation of the partial region of the cavity,
wherein the projection apparatus is configured to project a pattern intended for active triangulation onto a surface of the partial region of the cavity, and
wherein the imaging channel comprises a camera, and
wherein the camera, the objective lens, and the optical deflection apparatus are integrated in a chip.

2. The endoscope of claim 1, wherein the first optical deflection apparatus is arranged at a distal end of the endoscope.

3. (canceled)

4. The endoscope of claim 1, wherein the imaging channel comprises at least one lens.

5. The endoscope of claim 1, comprising at least one relay lens.

6. The endoscope of claim 1, wherein the first optical deflection apparatus has a parallelepiped form.

7. The endoscope of claim 1, wherein the first optical deflection apparatus has at least two mirrored internal surfaces.

8. (canceled)

9. The endoscope of claim 1, wherein the projection apparatus comprises a diffractive optical element for producing the pattern.

10. The endoscope of claim 1, wherein the pattern is a color-coded color pattern.

11. The endoscope of claim 1, wherein the projection channel is optically coupled to a light source.

12. The endoscope of claim 1, comprising an instrumentation channel.

13-15. (canceled)

16. The endoscope of claim 1, wherein the endoscope has an observation angle of 30°.

17. An endoscope for determining the depth of a partial region of a cavity, the endoscope comprising:
a first imaging channel having a first optical axis,
a first optical deflection apparatus arranged in the first imaging channel, the first optical deflection apparatus configured to cause a shift of the first optical axis in a direction transverse parallel with respect to the first optical axis;
wherein the imaging channel has an objective lens that comprises the first optical deflection apparatus;
a projection channel having a projection apparatus for active triangulation of the partial region of the cavity,
wherein the projection apparatus is configured to project a pattern intended for active triangulation onto a surface of the partial region of the cavity, and
wherein the imaging channel comprises a camera, and
wherein the camera, the objective lens, and the optical deflection apparatus are integrated in a chip;
a second imaging channel having a second optical axis; and
a second optical deflection apparatus arranged within the second imaging channel, the second optical deflection apparatus being configured to cause a shift of the second optical axis in a direction transverse parallel with respect to the second optical axis.

18. The endoscope of claim 17, wherein the direction of the transverse parallel shift of the second optical axis is opposite of the direction of the transverse parallel shift of the first optical axis.

19. The endoscope of claim 17, wherein the direction of the transverse parallel shift of the second optical axis is opposite of the direction of the transverse parallel shift of the first optical axis.

20. The endoscope of claim 17, wherein the second imaging channel is configured as a projection channel.
